(19) United States
(12) Patent Application Publication
(10) Pub. No.: US 2014/0023606 A1
(43) Pub. Date: Jan. 23, 2014

(54) PROCESS FOR PRODUCING A CONDITIONING CLEANING AGENT

(71) Applicant: Henkel AG & Co. KGaA, Duesseldorf (DE)

(72) Inventors: Volker Scheunemann, Lueneburg (DE); Erik Schulze zur Wiesche, Hamburg (DE); Dirk Hentrich, Hamburg (DE)

(73) Assignee: Henkel AG & Co. KGaA, Duesseldorf (DE)

(21) Appl. No.: 14/036,090

(22) Filed: Sep. 25, 2013

Related U.S. Application Data
(63) Continuation of application No. PCT/EP2012/054791, filed on Mar. 19, 2012.

(30) Foreign Application Priority Data
Mar. 25, 2011 (DE) 10 2011 015 192.3

(51) Int. Cl. A61K 8/34; A61Q 5/02; A61Q 5/12

(52) U.S. Cl. CPC A61K 8/345; A61Q 5/12; A61Q 5/02
USPC 424/70.13

(57) ABSTRACT

A process for producing a conditioning cleaning agent includes a) providing a microemulsion including (i) at least one alkyl(oligo)glycoside, (ii) at least one ester of glycerin with at least one C₁₀-C₂₄ fatty acid, (iii) at least one oil which is different from (ii), and (iv) water, and b) mixing the microemulsion with a cosmetic carrier that includes at least one polyhydroxy compound of formula (I) as defined in the specification herein.

### FIELD OF THE INVENTION

The present invention generally relates to cosmetics, and more particularly relates to a method for producing a conditioning cleaning agent, in which a microemulsion is mixed with a cosmetic carrier containing a special polyhydroxy compound.

The invention further relates to a conditioning cleaning agent which includes a microemulsion and a polyhydroxy compound, and to the use of the conditioning cleaning agent for strengthening the hair structure, for improving the sensory properties of hair and for increasing the hair volume.

### BACKGROUND OF THE INVENTION

Cosmetic hair cleaning agents have been known for a long time and are regularly improved or adapted to the changing needs of consumers.

For example, consumers expect a modern hair cleaning agent to leave behind a long-lasting, haptically and optically perceptible conditioning effect on the cleaned hair so that, for reasons of time, costs and environmental concerns, no hair after-treatment agent has to be applied.

Cleaning is generally understood to mean the freeing of hair from undesirable odors, dirt, dandruff, sebum deposits and/or residues of styling agents.

The term “haptically and optically perceptible conditioning effect” is understood to mean that the hair is smooth, easy to comb, soft, shiny and easy to style after the treatment (cleaning). Furthermore, cleaned hair should have increased volume.

It is known to add hair-conditioning active substances, such as e.g. silicones, oils or waxes, to hair cleaning agents to improve the conditioning.

However, silicone-based hair cleaning agents often have the disadvantage that, with regular use over a prolonged period, they make the hair feel undesirably heavy. Fine or damaged hair in particular loses its volume as a result.

The effectiveness of oils and waxes in hair cleaning agents is not as marked as that of the silicones. Moreover, oils and waxes can only be stabilized in hair cleaning agents in relatively small quantities, which makes the production of such agents more difficult.

Thus, to stabilize oil and wax components (or silicones) in cosmetic cleaning agents, it is necessary either to pass through process steps having a high energy requirement or to incorporate additional synthetic stabilizing agents into the cleaning agents, making the production of the agents disadvantageous from an economic and environmental point of view.

The need therefore still exists for cleaning agents which are obtainable by means of a simple production method, and which offer a conditioning advantage for optically and haptically unattractive hair.

The present invention was based on the object of providing an uncomplicated method for producing a conditioning cleaning agent.

The cleaning agent should contain relatively large quantities of at least one hair-conditioning lipid component, without steps having a high energy requirement, such as heating, melting or predispersing, being needed for stabilizing the lipid component in the cleaning agent. There should likewise be no need to incorporate polymeric or crystalline agents for stabilizing the lipid component.

Hair that has been damaged in its structure particularly as a result of chemical treatments or excessive exposure to UV light should be strengthened again by the application of the cleaning agent and should exhibit improved haptic properties, such as increased flexibility and a soft feel.

Fine, thin hair should exhibit increased hair volume after application of the cleaning agents.

A further object of the invention was to produce transparent cleaning agents.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method for producing a conditioning cleaning agent, comprising the following steps: providing a microemulsion containing (i) at least one alkyl (oligo)glycoside, (ii) at least one ester of glycerol with at least one C₁₀-C₂₄ fatty acid, (iii) at least one oil—which is different from (ii)—and (iv) water, and mixing the microemulsion with a cosmetic carrier, which contains at least one polyhydroxy compound of the following formula (I),

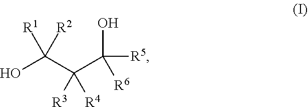

in which the residues R¹, R² independently of one another denote a hydrogen atom, a C₁-C₄ alkyl group or a phenyl group or in each case alkylene groups, which are constituents of a cyclopentane or cyclohexane ring; the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring; the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups, with the proviso that the residues R³, R⁴ do not simultaneously denote a hydroxyl group.

A conditioning cleaning agent, containing a microemulsion, which contains (i) at least one alkyl (oligo)glycoside, (ii) at least one ester of glycerol with at least one C₁₀-C₂₄ fatty acid, (iii) at least one oil—which is different from (ii)—and (iv) water, and at least one polyhydroxy compound of the following formula (I),

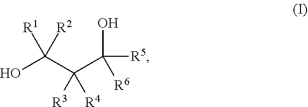

in which the residues R¹, R² independently of one another denote a hydrogen atom, a C₁-C₄ alkyl group or a phenyl group or in each case alkylene groups, which are constituents of a cyclopentane or cyclohexane ring; the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring; the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups, with the proviso that the residues R³, R⁴ do not simultaneously denote a hydroxyl group.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The invention provides a method for producing a conditioning cleaning agent, comprising the following steps:
a) providing a microemulsion containing
   (i) at least one alkyl (oligo)glycoside,
   (ii) at least one ester of glycerol with at least one C₁₀-C₂₄ fatty acid,
   (iii) at least one oil—which is different from (ii)—and
   (iv) water, and
b) mixing the microemulsion with a cosmetic carrier, which contains at least one polyhydroxy compound of the following formula (I),

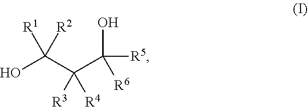

in which
   the residues R¹, R² independently of one another denote a hydrogen atom, a C₁-C₄ alkyl group or a phenyl group or in each case alkylene groups, which are constituents of a cyclopentane or cyclohexane ring,
   the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring,
   the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups,
      with the proviso that the residues R³, R⁴ do not simultaneously denote a hydroxyl group.

A cosmetic carrier is preferably understood to be an aqueous or aqueous-alcoholic carrier.

The cosmetic carrier preferably contains at least 40 wt. % water.

Furthermore, the cosmetic carrier can contain 0.01 to 40 wt. %, preferably 0.05 to 35 wt. % and in particular 0.1 to 30 wt. % of at least one alcohol, which can be selected from ethanol, 1-propanol, 2-propanol, isopropanol, 1-butanol, 2-butanol, 1-pentanol, 2-pentanol, 1-hexanol, 2-hexanol, benzyl alcohol, phenoxyethanol or mixtures of these alcohols.

The water-soluble alcohols are preferred.

Particularly preferred are ethanol, 1-propanol, 2-propanol, isopropanol, benzyl alcohol and/or phenoxyethanol and mixtures of these alcohols.

The method according to the invention requires no particular order in the mixing of the components a) and b). In principle, it is possible first to present a polyhydroxy compound according to formula (I) in a suitable carrier and then to add the microemulsion thereto. It is likewise possible to add a carrier containing at least one polyhydroxy compound according to formula (I) to the microemulsion.

In a preferred embodiment, a cosmetic carrier as described above is first presented. All of the optional components of the cleaning agent and at least one polyhydroxy compound according to formula (I) are then incorporated into the carrier, it being preferred if all of the steps are carried out at ambient temperature by mixing (in particular by gently stirring) the respective component with the carrier.

After the addition of the microemulsion, which preferably also takes place at ambient temperature by stirring the microemulsion into the carrier described above, the pH value and the viscosity of the cleaning agent are adjusted to the desired values in each case.

Suitable polyhydroxy compounds according to formula (I) are used in the method according to the invention preferably in a quantity of 0.01 to 10 wt. %, more preferably 0.05 to 7.5 wt. %, particularly preferably 0.075 to 5 wt. % and in particular 0.1 to 3 wt. %, the quantitative data being based on the total weight of the conditioning cleaning agent.

Preferred polyhydroxy compounds according to formula (I) are those in which
the residues R¹, R² independently of one another denote a hydrogen atom or a methyl group,
the residues R³, R⁴ independently of one another denote a hydrogen atom, a hydroxyl group or a methyl group, with the proviso that R³, R⁴ do not simultaneously denote a hydroxyl group,
the residues R⁵, R⁶ independently of one another denote a hydrogen atom or a methyl group, or one of the residues R⁵, R⁶ denotes a hydrogen atom or a methyl group and the other residue denotes one of the following groupings

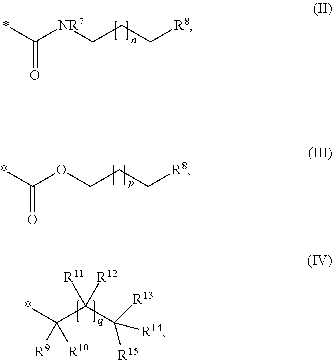

in which
   R⁷ denotes a hydrogen atom or a C₁-₄ alkyl group,
   R⁸ denotes a hydrogen atom, a methyl group or a hydroxyl group,
   n, p, q independently of one another denote numbers from 0 to 10, and
   the residues R⁹ to R¹⁵ independently of one another denote a hydrogen atom, a C₁₄ alkyl group or a C₁₄ hydroxyl group, with the proviso that each C atom carries no more than one hydroxyl group.

Particularly preferred polyhydroxy compounds according to formula (I) are 1,3-propylene glycol, glycerol, erythritol, pentaerythritol, sorbitol, mannitol, xylitol and/or panthenol, it being likewise particularly preferred to use mixtures of two or more of the above-mentioned polyhydroxy compounds in the method according to the invention.

Particularly preferred are panthenol, glycerol and/or sorbitol.

Microemulsions a) that are suitable for use in the method according to the invention preferably have an average particle size by volume of less than 3 μm, more preferably less than 2 μm and in particular less than 1 μm.

They contain—based on their total weight—preferably
(i) 1 to 40 wt. %, more preferably 5 to 30 wt. % and in particular 10 to 20 wt. % of at least one alkyl (oligo)glycoside of the general formula RO-[G]ₓ, in which R denotes an alkyl and/or alkenyl residue with 4 to 22 C atoms, G denotes a sugar residue with 5 or 6 C atoms and x denote numbers from 1 to 10,
(ii) 1 to 15 wt. %, more preferably 2 to 12.5 wt. % and in particular 4 to 10 wt. % of at least one saturated or unsaturated, branched or unbranched monoester and/or diester of glycerol with a C₁₀-C₂₄ fatty acid,
(iii) 5 to 45 wt. %, more preferably 7.5 to 40 wt. % and in particular 10 to 30 wt. % of at least one oil and
(iv) 40 to 80 wt % water.

Particularly suitable alkyl (oligo)glycosides (i) are derived from aldoses and/or ketoses with 5 or 6 carbon atoms, preferably from glucose.

The residue R particularly preferably denotes an alkyl residue with 6 to 20 and in particular with 8 to 18 carbon atoms.

The index x in the general formula RO-[G]ₓ denotes the degree of oligomerization (DP), i.e. the distribution of the mono- and oligoglycosides. The index x preferably has a value in the range of 1 to 6, particularly preferably in the range of 1 to 3, wherein it can be not an integer but a fractional number which can be determined analytically. Particularly preferred alkyl (oligo)glycosides have a degree of oligomerization of between 1.2 and 1.5.

Particularly suitable alkyl (oligo)glycosides are known and are commercially available with the INCI names Decyl Glucoside, Lauryl Glucoside and Coco Glucoside from various suppliers.

Particularly suitable esters (ii) are monoesters of glycerol with linear fatty acids having alkyl chain lengths of 12 to 22 C atoms. Examples of particularly suitable esters (ii) are glyceryl monolaurate, glyceryl monomyristate, glyceryl monopalmitate, glyceryl monostearate and/or glyceryl monooleate. Glyceryl monooleate is particularly suitable.

Suitable oils (iii) can be selected from mineral, natural and synthetic oil components and/or fatty substances.

As natural (vegetable) oils, triglycerides and mixtures of triglycerides can be used. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and shea butter.

In particular, mineral oils, paraffin and isoparaffin oils and synthetic hydrocarbons are used as mineral oils. An example of a hydrocarbon that can be used is e.g. 1,3-di-(2-ethylhexyl)cyclohexane (Cetiol® S), which is available as a commercial product.

Silicone compounds are suitable as synthetic oils.

Silicones produce excellent conditioning properties on the hair. In particular, they produce better combability of the hair in the wet and dry state and in many cases have a positive effect on hair feel and the softness of hair.

Suitable silicones can be selected from among:
(i) polyalkyl siloxanes, polyaryl siloxanes, polyalkylaryl siloxanes, which are volatile or non-volatile, straight-chained, branched or cyclic, crosslinked or non-crosslinked;
(ii) polysiloxanes, which contain in their general structure one or more organofunctional groups that are selected from among:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)-polyoxyalkylene(B) block copolymers of the (A-B)ₙ type with n>3;
(iv) grafted silicone polymers with a non-silicone-containing, organic backbone, which consist of an organic main chain which is formed from organic monomers containing no silicone, onto which at least one polysiloxane macromer has been grafted in the chain and optionally on at least one end of the chain;
(v) grafted silicone polymers with a polysiloxane backbone, onto which non-silicone-containing, organic monomers have been grafted, having a polysiloxane main chain onto which at least one organic macromer which contains no silicone has been grafted in the chain and optionally on at least one end thereof;
(vi) or mixtures thereof.

A diallyl ether can also be used as an oil component.

Dialkyl ethers that can be used are in particular di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert.-butyl ether, di-isopentyl ether, di-3-ethyldecyl ether, tert.-butyl-n-octyl ether, isopentyl-n-octyl ether and 2-methylpentyl-n-octyl ether.

Particularly preferred is di-n-octyl ether, which is commercially available with the name Cetiol® OE.

Fatty substances are to be understood as fatty acids, fatty alcohols and natural and synthetic waxes, which may be present both in solid form and as a liquid in aqueous dispersion.

As fatty acids, it is possible to use linear and/or branched, saturated and/or unsaturated fatty acids with -30 carbon atoms. Preferred are fatty acids with 10-22 carbon atoms. Among these, e.g. the isostearic acids, such as the commercial products Emersol® 871 and Emersol® 875, and isopalmitic acids, such as the commercial product Edenor® IP 95, and all other fatty acids marketed under the trade names Edenor® (Cognis) should be mentioned. Other typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof.

Particularly preferred are generally the fatty acid blends that are obtainable from coconut oil or palm oil; the use of stearic acid is usually particularly preferred.

As fatty alcohols, it is possible to use saturated, mono- or polyunsaturated, branched or unbranched fatty alcohols with C₆-C₃₀, preferably C₁₀-C₂₂ and most particularly preferably C₁₂-C₂₂ carbon atoms. For example, decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol and the Guerbet alcohols thereof can be used, this list being intended to be of an exemplary and non-limiting nature. However, the fatty alcohols are derived from preferably natural fatty acids, wherein it can generally be assumed that they are obtained from the esters of the fatty acids by reduction. According to the invention, it is also possible to use those fatty alcohol blends that are produced by reduction of naturally occurring triglycerides, such as beef tallow, palm oil, ground nut oil, rapeseed oil, cottonseed oil, soybean oil, sunflower oil and linseed oil or fatty acid esters formed from the transesterification products thereof with corresponding alcohols, and thus represent a mixture of different fatty alcohols. Substances of this type can be purchased e.g. with the names Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to use wool wax alcohols, as can be purchased e.g. with the names Corona®, White Swan®, Coronet® or Fluilan®.

As natural or synthetic waxes, it is possible to use solid paraffins or isoparaffins, carnauba waxes, beeswaxes, candelilla waxes, ozokerites, ceresin, cetaceum, sunflower wax, fruit waxes, such as e.g. apple wax or citrus wax, micro waxes comprising PE or PP. Waxes of this type are available e.g. through Kahl & Co., Trittau.

Other fatty substances are e.g.
ester oils. Ester oils are to be understood as the esters of C₆-C₃₀ fatty acids with C₂-C₃₀ fatty alcohols. Preferred are the monoesters of fatty acids with alcohols having 2 to 24 C atoms. Examples of fatty acid portions that can be used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and rucic acid and technical mixtures thereof.
Examples of the fatty alcohol portions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V).
dicarboxylic acid esters, such as di-n-butyl adipate, di-(2-ethylhexyl) adipate, di-(2-ethylhexyl) succinate and diisotridecyl acetate as well as diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,
symmetric, asymmetric or cyclic esters of carbonic acid with fatty alcohols,
glycerol carbonate or dicaprylyl carbonate (Cetiol® CC),
ethoxylated or non-ethoxylated mono-, di- and trifatty acid esters of saturated and/or unsaturated, linear and/or branched fatty acids with glycerol, such as e.g. Monomuls® 90-018, Monomuls® 90-L12, Cetiol® HE or Cutina® MD.

Another essential ingredient of the microemulsions a) is water. The microemulsion a) contains—based on its total weight—preferably 40 to 80 wt. %, more preferably 40 to 70 wt. % and in particular 45 to 65 wt. % water.

The microemulsion a) can be produced (preferably before carrying out the method according to the invention) preferably by mixing the liquid oil phases (ii) and (iii) with the surfactant-containing aqueous phase ((i) and (iv)) with stirring.

Alternatively, the microemulsion a) can also be used in the method according to the invention as a ready-made commercial product. An example of a suitable, commercially available microemulsion a) is the microemulsion available with the name “Plantasil Micro®” from Cognis.

The microemulsion a) is preferably used in the method according to the invention in a quantity of 0.01 to 50 wt. %, more preferably 0.1 to 30 wt. %, particularly preferably 0.5 to 20 wt. % and in particular 1 to 15 wt. %, the quantities being based on the total weight of the conditioning cleaning agent.

In a particularly preferred embodiment, the production method according to the invention leads to conditioning cleaning agents that are transparent.

The term “transparent” is understood to mean that the conditioning cleaning agents have an NTU (nephelometric turbidity unit) value of no more than 100, preferably no more than 50 and in particular no more than 30.

For some cosmetic forms of application, it may be preferred to produce conditioning cleaning agents that give outstanding cleaning, are very mild and exhibit particularly good foaming power.

In order to achieve these goals, it is advantageous if the method according to the invention passes through production steps which contain the incorporation of further surfactants.

Suitable further surfactants can be contained in the microemulsion a), or they can be added to the cosmetic carrier before or after the incorporation of the microemulsion.

In the event that the further surfactants are components of the microemulsion a), they are added to the surfactant-containing aqueous phase ((i) and (iv)) of the microemulsion before mixing with the oil phases ((ii) and (iii)).

The microemulsion a) preferably contains no more than 25 wt. % of further surfactants.

Suitable surfactants for the method according to the invention can be selected from mild anionic, amphoteric/zwitterionic and/or nonionic surfactants with good foaming properties.

Suitable anionic surfactants can be used in the method according to the invention preferably in quantities of 0.1 to 40 wt. %, more preferably 0.5 to 30 wt. %, particularly preferably 1 to 25 wt. % and in particular 3 to 20 wt. %, the quantitative data being based on the total weight of the conditioning cleaning agent.

The suitable anionic surfactants include:
linear and branched fatty acids with 8 to 30 C atoms (soaps),
ether carboxylic acids of the formula R—O—(CH₂-CH₂O)ₓ—CH₂—COOH, in which R is a linear or branched, saturated or unsaturated alkyl group with 8 to 30 C atoms and x=0 or 1 to 16,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono- and/or dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
alpha-olefin sulfonates with 8 to 24 C atoms,
alkyl sulfate and/or alkyl polyglycol ether sulfate salts of the formula R—O—(CH₂-CH₂O)ₓ—OSO₃⁻X⁺, in which R is a preferably linear or branched, saturated or unsaturated alkyl group with 8 to 30 C atoms, x =0 or 1 to 12 and X is an alkali or ammonium ion,
sulfonates of unsaturated fatty acids with 8 to 24 C atoms and 1 to 6 double bonds,
esters of tartaric acid and citric acid with alcohols, which represent addition products of about 2-15 molecules ethylene oxide and/or propylene oxide to fatty alcohols with 8 to 22 C atoms,
alkyl and/or alkenyl ether phosphates of the formula,

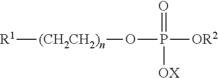

in which R¹ preferably denotes an aliphatic hydrocarbon residue with 8 to 30 carbon atoms, R² denotes hydrogen, a (CH₂CH₂O)ₙR¹ residue or X, n denotes numbers from 0 to 10 and X denotes hydrogen, an alkali or alkaline earth metal or NR³R⁴R⁵R⁶, with R³ to R⁶ independently of one another denoting a C₁ to C₄ hydrocarbon residue.

Preferred anionic surfactants are ether carboxylic acids of the above-mentioned formula, acyl sarcosides with 8 to 24 C atoms in the acyl group, sulfosuccinic acid mono- and/or diallyl esters with 8 to 24 C atoms in the alkyl group and sulfosuccinic acid monoalkyl polyoxyethyl esters with 8 to 24 C atoms in the alkyl group and 1 to 6 oxyethyl groups, alpha-olefin sulfonates with 8 to 24 C atoms and/or alkyl sulfate and/or alkyl polyglycol ether sulfate salts of the above-mentioned formula.

Particularly preferred anionic surfactants are straight-chained or branched alkyl ether sulfates containing an alkyl residue with 8 to 18 and in particular with 10 to 16 C atoms and 1 to 6 and in particular 2 to 4 ethylene oxide units.

Further particularly preferred anionic surfactants are straight-chained or branched alkyl sulfonates containing an alkyl residue with 8 to 18 and in particular with 10 to 16 C atoms. Particularly preferred are the sodium, magnesium and/or triethanolamine salts of linear or branched lauryl, tridecyl and/or myristyl sulfate having a degree of ethoxylation of 2 to 4.

Suitable amphoteric/zwitterionic surfactants can be used in the method according to the invention preferably in quantities of 0 to 20 wt. %, more preferably 0.25 to 17.5 wt. %, particularly preferably 0.5 to 15 wt. % and in particular 1 to 10 wt. %, the quantitative data being based on the total weight of the conditioning cleaning agent.

Suitable amphoteric/zwitterionic surfactants can be selected from compounds of the following formulae (i) to (v), in which the residue R in each case denotes a straight-chained or branched, saturated or mono- or polyunsaturated alkyl or alkenyl residue with 8 to 24 carbon atoms,

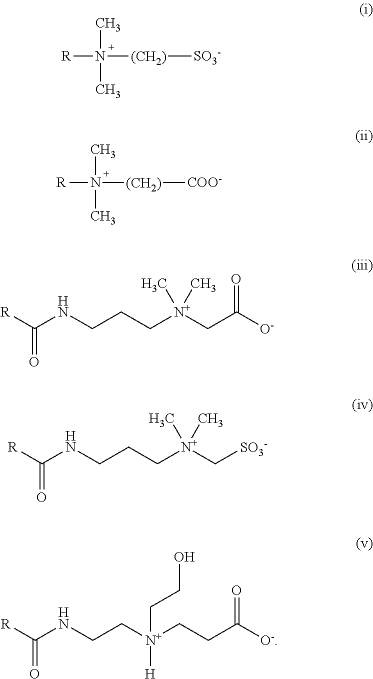

Particularly suitable amphoteric/zwitterionic surfactants are alkylamidoalkyl betaines and/or alkyl ampho(di)acetates of the above-mentioned formulae (i) to (v).

The particularly suitable amphoteric/zwitterionic surfactants include the surfactants known by the INCI name Cocamidopropyl Betaine and Disodium Cocoamphodiacetate.

Suitable nonionic surfactants can be used in the method according to the invention (in addition to the alkyl (oligo)glycoside(s) in the microemulsion a)) preferably in quantities of 0 to 20 wt. %, more preferably 0.25 to 17.5 wt. %, particularly preferably 0.5 to 15 wt. % and in particular 1 to 10 wt. %, the quantitative data being based on the total weight of the conditioning cleaning agent.

The suitable nonionic surfactants/emulsifiers include e.g.
C₈-C₃₀ fatty acid mono- and diesters of addition products of 1 to 30 moles ethylene oxide to glycerol,
amine oxides,
addition products of 2 to 50 moles ethylene oxide and/or 0 to 5 moles propylene oxide to linear and branched fatty alcohols with 8 to 30 C atoms, to fatty acids with 8 to 30 C atoms and to alkyl phenols with 8 to 15 C atoms in the alkyl group,
sorbitan fatty acid esters and addition products of ethylene oxide to sorbitan fatty acid esters, such as e.g. polysorbates,
sugar fatty acid esters and addition products of ethylene oxide to sugar fatty acid esters,
addition products of ethylene oxide to fatty acid alkanolamides and fatty amines and/or
alkyl polyglucosides.

In the event that a nonionic surfactant is used as a further surfactant in the method according to the invention, alkyl oligoglucosides, in particular alkyl oligoglucosides based on hydrogenated C_{12/14} coconut alcohol with a DP of 1-3, as are commercially available e.g. with the INCI name “Coco-Glucoside”, are preferred.

Further preferred nonionic surfactants are the C₈-C₃₀ fatty acid mono- and diesters of addition products of 1 to 30 moles ethylene oxide to glycerol. Particularly preferred are the C₁₀-C₁₆ fatty acid mono- and diesters of addition products of 1 to 10 moles ethylene oxide to glycerol. Preferred is in particular PEG-7 Glyceryl Cocoate known by the INCI name.

In a first particularly preferred embodiment of the invention, the method for producing the conditioning cleaning agent encompasses the following steps:
a) providing a microemulsion, containing
   (i) at least one alkyl (oligo)glycoside,
   (ii) at least one ester of glycerol with at least one C₁₀-C₂₄ fatty acid,
   (iii) at least one oil—which is different from (ii)—and
   (iv) water, and
b) mixing the microemulsion with a cosmetic carrier, which contains at least one polyhydroxy compound according to the above-mentioned formula (I) and at least one anionic surfactant.

Within this embodiment it is particularly preferred if the cosmetic carrier contains
0.01 to 10 wt. % 1,3-propylene glycol, glycerol, erythritol, pentaerythritol, sorbitol, mannitol, xylitol and/or panthenol, and
1 to 25 wt. % of at least one anionic surfactant, which can be selected from the group of the ether carboxylic acids, the acyl sarcosides with 8 to 24 C atoms in the acyl group, the sulfosuccinic acid mono- and/or dialkyl esters with 8 to 24 C atoms in the alkyl group and the sulfosuccinic acid monoalkyl polyoxyethyl esters with 8 to 24 C atoms in the alkyl group and 1 to 6 oxyethyl groups, the alpha-olefin sulfonates with 8 to 24 C atoms and/or the alkyl sulfate and/or alkyl polyglycol ether sulfate salts.

Within this embodiment it is preferred in particular if the cosmetic carrier contains
0.05 to 7.5 wt. % glycerol, sorbitol and/or panthenol and
3 to 20 wt % straight-chained or branched alkyl ether sulfates having an alkyl residue with 8 to 18 and in particular with 10 to 16 C atoms as well as 1 to 6 and in particular 2 to 4 ethylene oxide units.

In a second particularly preferred embodiment of the invention, the method for producing the conditioning cleaning agent encompasses the following steps:
a) providing a microemulsion containing
   (i) at least one alkyl (oligo)glycoside,
   (ii) at least one ester of glycerol with at least one C₁₀-C₂₄ fatty acid,
   (iii) at least one oil—which is different from (ii)—and
   (iv) water, and
b) mixing the microemulsion with a cosmetic carrier, which contains at least one polyhydroxy compound according to the above-mentioned formula (I), at least one anionic surfactant, at least one amphoteric/zwitterionic surfactant and at least one nonionic surfactant.

Within this embodiment it is particularly preferred if the cosmetic carrier contains
0.01 to 10 wt. % 1,3-propylene glycol, glycerol, erythritol, pentaerythritol, sorbitol, mannitol, xylitol and/or panthenol,
1 to 25 wt. % of at least one anionic surfactant, which can be selected from the group of the ether carboxylic acids of the above-mentioned formula, the acyl sarcosides with 8 to 24 C atoms in the acyl group, the sulfosuccinic acid mono- and/or dialkyl esters with 8 to 24 C atoms in the alkyl group and the sulfosuccinic acid monoalkyl polyoxyethyl esters with 8 to 24 C atoms in the alkyl group and 1 to 6 oxyethyl groups, the alpha-olefin sulfonates with 8 to 24 C atoms and/or the alkyl sulfate and/or alkyl polyglycol ether sulfate salts,
0.25 to 17.5 wt. % of at least one amphoteric/zwitterionic surfactant, which can be selected from alkylamidoalkyl betaines and/or alkyl ampho(di)acetates of the above-mentioned formulae (i) to (v) and
0.25 to 17.5 wt. % of at least one nonionic surfactant, which can be selected from C₈-C₃₀ fatty acid mono- and diesters of addition products of 1 to 30 moles ethylene oxide to glycerol, amine oxides, addition products of 2 to 50 moles ethylene oxide and/or 0 to 5 moles propylene oxide to linear and branched fatty alcohols with 8 to 30 C atoms, to fatty acids with 8 to 30 C atoms and to alkylphenols with 8 to 15 C atoms in the alkyl group, sorbitan fatty acid esters and addition products of ethylene oxide to sorbitan fatty acid esters, such as e.g. polysorbates, sugar fatty acid esters and addition products of ethylene oxide to sugar fatty acid esters, addition products of ethylene oxide to fatty acid alkanolamides and fatty amines and/or alkyl polyglucosides.

Within this embodiment it is particularly preferred if the cosmetic carrier contains
0.05 to 7.5 wt. % glycerol, sorbitol and/or panthenol,
3 to 20 wt. % straight-chained or branched alkyl ether sulfates, having an alkyl residue with 8 to 18 and in particular with 10 to 16 C atoms and 1 to 6 and in particular 2 to 4 ethylene oxide units,
1 to 10 wt. % of at least one of the surfactants known by the INCI name Cocamidopropyl Betaine and Disodium Cocoamphodiacetate and
1 to 10 wt. % C₈-C₃₀ fatty acid mono- and/or diesters of addition products of 1 to 30 moles ethylene oxide to glycerol, addition products of 2 to 50 moles ethylene oxide to linear and branched fatty alcohols with 8 to 30 C atoms and/or alkyl polyglucosides.

The conditioning effect of the conditioning cleaning formulation produced by the method according to the invention is achieved through the fact that the lipid component(s) from the microemulsion is (are) distributed and deposited on the hair fibers homogeneously. As a result, the hair fibers are smoothed and, after the treatment with the conditioning cleaning formulations, they exhibit more flexibility and a soft feel.

The polyhydroxy compounds contribute to the smoothing of the hair fibers and the strengthening of the hair structure.

To support the depositing of the lipid component(s) on the hair fibers, it may be advantageous if production steps which contain the incorporation of cationic polymers are passed through in the method according to the invention.

Suitable cationic polymers can be contained in the microemulsion a) or they can be added to the cosmetic carrier before or after the incorporation of the microemulsion.

Suitable cationic polymers are used in the method according to the invention preferably in a quantity of 0.01 to 10 wt. %, preferably 0.05 to 5 wt. % and in particular 0.1 to 3 wt. %, the quantities stated being based on the total weight of the conditioning cleaning agent.

Suitable cationic polymers are e.g.:
quaternized cellulose derivatives, as are commercially available with the names Celquat® and Polymer JR®,
hydrophobically modified cellulose derivatives, e.g. the cationic polymers marketed with the trade name SoftCat®,
cationic alkyl polyglycosides,
cationized honey, e.g. the commercial product Honeyquat® 50,
cationic guar derivatives, such as in particular the products marketed with the trade names Cosmedia®Guar and Jaguar®,
polymeric dimethyl diallyl ammonium salts and copolymers thereof with esters and amides of acrylic acid and methacrylic acid. The products that are commercially available with the names Merquat®100 (poly(dimethyl diallyl ammonium chloride)) and Merquat®550 (dimethyl diallyl ammonium chloride-acrylamide copolymer) are examples of these cationic polymers,
copolymers of vinylpyrrolidone with quaternized derivatives of dialkylaminoalkyl acrylate and methacrylate, such as e.g. vinylpyrrolidone-dimethylaminoethyl methacrylate copolymers quaternized with diethyl sulfate. Compounds of this type are commercially available with the names Gafquat®734 and Gafquat®755,
vinylpyrrolidone-vinylimidazolium methochloride copolymers, as sold with the names Luviquat® FC 370, FC 550, FC 905 and HM 552,
quaternized polyvinyl alcohol,
   and the polymers known by the names
Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89.

Particularly preferred cationic polymers that can be used in the method according to the invention are quaternized cellulose polymers, cationic guar derivatives and/or acrylic acid (derivative)-based cationic polymers, which are selected in particular from the polymers known by the INCI names Guar Hydroxypropyltrimonium Chloride, Polyquaternium-6, Polyquaternium-7, Polyquaternium-10, Polyquaternium-37 and/or Polyquaternium-67.

In a third particularly preferred embodiment of the invention, the method for producing the conditioning cleaning agent encompasses the following steps:
a) providing a microemulsion containing
   (i) at least one alkyl (oligo)glycoside,
   (ii) at least one ester of glycerol with at least one C₁₀-C₂₄ fatty acid,
   (iii) at least one oil—which is different from (ii)—and
   (iv) water, and
b) mixing the microemulsion with a cosmetic carrier, which contains at least one protein hydrolyzate, at least one anionic surfactant and at least one cationic polymer.

Within this embodiment it is particularly preferred if the cosmetic carrier contains
0.01 to 10 wt. % 1,3-propylene glycol, glycerol, erythritol, pentaerythritol, sorbitol, mannitol, xylitol and/or panthenol,
1 to 25 wt. % of at least one anionic surfactant, which can be selected from the group of the ether carboxylic acids of the above-mentioned formula, the acyl sarcosides with 8 to 24 C atoms in the acyl group, the sulfosuccinic acid mono- and/or dialkyl esters with 8 to 24 C atoms in the alkyl group and the sulfosuccinic acid monoalkyl polyoxyethyl esters with 8 to 24 C atoms in the alkyl group and 1 to 6 oxyethyl groups, the alpha-olefin sulfonates with 8 to 24 C atoms and/or the alkyl sulfate and/or alkyl polyglycol ether sulfate salts, and
0.01 to 10 wt. % of at least one cationic polymer, selected from the group of the quaternized cellulose polymers, the cationic guar derivatives and/or the acrylic acid (derivative)-based cationic polymers.

Within this embodiment it is particularly preferred if the cosmetic carrier contains
0.05 to 7.5 wt. % glycerol, sorbitol and/or panthenol,
3 to 20 wt. % straight-chained or branched alkyl ether sulfates having an alkyl residue with 8 to 18 and in particular with 10 to 16 C atoms and 1 to 6 and in particular 2 to 4 ethylene oxide units, and
0.05 to 5 wt. % of at least one of the cationic polymers known by the INCI name Guar Hydroxypropyltrimonium Chloride, Polyquaternium-6, Polyquaternium-7, Polyquaternium-10, Polyquaternium-37 and/or Polyquaternium-67.

The cosmetic carrier described above can also contain a series of other optional active substances that can produce advantageous properties on the hair and do not make the method according to the invention more difficult. The preferred optional active substances include e.g.:
vitamins, vitamin derivatives and/or vitamin precursors, which can be used in the method according to the invention preferably in a quantity of 0.001 to 10 wt. %, more preferably 0.005 to 7.5 wt. % and in particular 0.01 to 5 wt. %,
active anti-dandruff substances, which can be used in the method according to the invention preferably in a quantity of 0.01 to 10 wt. %, more preferably 0.025 to 7.5 wt. %, particularly preferably 0.05 to 5 wt. % and in particular 0.075 to 3 wt. %, the quantitative data being based in each case on the total weight of the conditioning cleaning agent.

Suitable vitamins are preferably to be understood as the following vitamins, provitamins and vitamin precursors and derivatives thereof:

Vitamin A: the group of substances referred to as vitamin A includes retinol (vitamin A₁) and 3,4-didehydroretinol (vitamin A₂). β-Carotene is the provitamin of retinol. Suitable as a vitamin A component are e.g. vitamin A acid and esters thereof, vitamin A aldehyde and vitamin A alcohol and esters thereof, such as the palmitate and acetate.
Vitamin B: the vitamin B group or vitamin B complex includes inter alia
vitamin B₁ (thiamin)
vitamin B₂ (riboflavin)
vitamin B₃. The compounds nicotinic acid and nicotinamide (niacinamide) are often known by this name.
vitamin B₅ (pantothenic acid and panthenol). Within this group, panthenol is preferably used. Derivatives of panthenol that can be used are in particular the esters and ethers of panthenol and cationically derivatized panthenols. Individual representatives are e.g. panthenol triacetate, panthenol monoethyl ether and monoacetate thereof and cationic panthenol derivatives.
vitamin B₆ (pyridoxine together with pyridoxamine and pyridoxal).
   Vitamin C (ascorbic acid): use in the form of the palmitic acid ester, glucosides or phosphates may be preferred. Use in combination with tocopherols may likewise be preferred.
   Vitamin E (tocopherols, in particular a-tocopherol).
   Vitamin F: the term “vitamin F” is usually understood to mean essential fatty acids, in particular linoleic acid, linolenic acid and arachidonic acid.
   Vitamin H: the compound (3aS,4S,6aR)-2-oxohexahydrothienol[3,4-d]-imidazole-4 valeric acid is referred to as vitamin H, for which the trivial name biotin has now become accepted.

The use of the vitamins, provitamins and vitamin precursors from the groups A, B, E and H is preferred. Nicotinamide, biotin, pantolactone and/or panthenol are particularly preferred.

Suitable active anti-dandruff substances can be selected from piroctone olamine, climbazole, zinc pyrithione, ketoconazole, salicylic acid, sulfur, selenium sulfide, tar preparations, undecenoic acid derivatives, burdock root extracts, poplar extracts, nettle extracts, walnut shell extracts, birch extracts, willow bark extracts, rosemary extracts and/or arnica extracts.

Climbazole, zinc pyrithione and piroctone olamine are preferred.

Other active substances, auxiliary substances and additives that can be used in the method according to the invention, are e.g.:
plant extracts,
humectants,
perfumes,
UV filters,
thickeners, such as gelatin or vegetable gums, e.g. agar-agar, guar gum, alginates, xanthan gum, gum arabic, karaya gum, locust bean gum, flaxseed gums, dextrans, cellulose derivatives, e.g. methyl cellulose, hydroxyalkyl cellulose and carboxymethyl cellulose, starch fractions and derivatives, such as amylose, amylopectin and dextrins, clays and phyllosilicates, such as e.g. bentonite, or fully synthetic hydrocolloids, such as e.g. polyvinyl alcohol, the Ca, Mg or Zn soaps,
structurants, such as maleic acid and lactic acid,
dimethyl isosorbide,
cyclodextrins,
fiber structure improving active substances, in particular mono-, di- and oligosaccharides, such as e.g. glucose, galactose, fructose, fruit sugar and lactose,
colorants for coloring the agent,
substances for adjusting the pH value, e.g. α- and β-hydroxycarboxylic acids, such as citric acid, lactic acid, malic acid and glycolic acid,
active substances, such as bisabolol,
chelating agents, such as EDTA, NTA, β-alanine diacetic acid and phosphonic acids,
ceramides. Ceramides are understood to be N-acylsphingosine (fatty acid amides of sphingosine) or synthetic analogs of such lipids (so-called pseudo-ceramides),
propellants, such as propane-butane mixtures, N₂O, dimethyl ether, CO₂ and air,
antioxidants,
consistency enhancers, such as sugar esters, polyol esters or polyol alkyl ethers,
preservatives, such as e.g. sodium benzoate or salicylic acid,
viscosity regulators, such as salts (NaCI).

The method according to the invention is preferably suitable for producing conditioning cleaning agents having a pH value in the range of 1.5 to 7.5, preferably 2 to 6.5 and in particular 3 to 6.

The method according to the invention has the advantage that it is particularly simple to carry out and requires a low energy input. The conditioning components a) and b) can be mixed together in any order and further auxiliary substances and active substances can be incorporated into the cosmetic carrier without the method becoming noticeably more complicated as a result.

Furthermore, with the method according to the invention, conditioning cleaning agents can be produced having a high proportion of lipids (for particularly good hair conditioning), which also foam and clean well and in addition are transparent and stable.

Hair treatment with the conditioning cleaning agents that can be obtained by the method according to the invention leads to strengthened hair having a soft feel, increased flexibility and more volume.

The invention secondly provides a conditioning cleaning agent which contains in a cosmetic carrier
a) a microemulsion containing
   (i) at least one alkyl (oligo)glycoside,
   (ii) at least one ester of glycerol with at least one C₁₀-C₂₄ fatty acid,
   (iii) at least one oil—which is different from (ii)—and
   (iv) water, and
b) at least one polyhydroxy compound of the following formula (I),

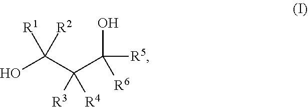

in which
   the residues R¹, R² independently of one another denote a hydrogen atom, a C₁-C₄ alkyl group or a phenyl group or in each case alkylene groups, which are constituents of a cyclopentane or cyclohexane ring,
   the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring,
   the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups,
with the proviso that the residues R³, R⁴ do not simultaneously denote a hydroxyl group.

The invention thirdly provides the use of the cosmetic cleaning agent described above for strengthening the hair structure, for improving the sensory properties of hair and for increasing hair volume.

With regard to further preferred embodiments of the agent according to the invention and the use according to the invention, statements made regarding the method according to the invention apply mutatis mutandis.

### EXAMPLES

Conditioning hair shampoos

| | 1 | 2 | 3 | 4 |
|---|---|---|---|---|
| Texapon ®¹ N70 NA | 15 | 12 | 10 | 15 |
| Dehyton ®² K | 10 | | | 10 |
| Dehyton ®³ G | | 10 | | |
| Plantacare ®⁴ 818 UP | 4 | | 2 | 4 |
| Nicotinamide | 0.3 | | 0.1 | |
| Panthenol | 0.5 | | | 0.2 |
| Glycerol | | 1 | | 0.5 |
| Sorbitol | | | 1.5 | |
| Plantasil ®⁵ Micro | 7 | 10 | 15 | 7 |
| Cutina ®⁶ HR | 0.4 | 0.3 | 0.6 | 0.4 |
| Cetiol ®⁷ HE | 1 | 0.8 | 0.4 | 1 |
| Polymer JR ®⁸ 400 | 0.3 | | 0.5 | 0.3 |
| Polyquaternium-7 | | 0.15 | | |
| Arlypon ®⁹ F | 1.2 | | 1 | 1.2 |
| Dow Corning ®¹⁰ 200 | | | 0.1 | |
| Citric acid | 0.1-1.5 | 0.1-1.5 | 0.1-1.5 | 0.1-1.5 |
| Zinc pyrithione | | 1 | 0.5 | |
| Preservative, perfume | q.s. | q.s. | q.s. | q.s. |
| Water | to 100 | to 100 | to 100 | to 100 |

In the above-mentioned hair shampoos, the following commercial products were used:
¹INCI name: Sodium Laureth Sulfate; AS 68-73%; Cognis
²INCI name: Cocamidopropyl Betaine; AS 29-32%; Cognis
³INCI name: Disodium Cocoamphodiacetate; AS about 30%; Cognis
⁴INCI name: Coco Glucoside; AS 51-53%; Cognis
⁵INCI name: Aqua, Dicaprylyl Ether, Decyl Glucoside, Glyceryl Oleate; Cognis
⁶INCI name: Hydrogenated Castor Oil; Cognis
⁷INCI name: PEG-7 Glyceryl Cocoate; Cognis
⁸INCI name: Polyquatemium-10; Dow
⁹INCI name: Laureth-2; Cognis
¹⁰INCI name: Dimethylpolysiloxane; Dow Corning

The conditioning hair shampoos of examples 1 to 4 were produced by the following method:
the surfactants, the polyhydroxy compounds and optionally nicotinamide were mixed at ambient temperature with half of the water,
the cationic polymer(s) was (were) pre-swollen in water at ambient temperature and then added to the surfactant mixture,
the remaining active substances (except the citric acid and the Arlypon F) were added to the batch consecutively at ambient temperature with stirring, with the formation of a transparent cleaning composition,
the pH value was adjusted with citric acid to a value of about 4.5 to 5.5,
the desired viscosity in each case was adjusted with Arlypon F.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for producing a conditioning cleaning agent, comprising the following steps:
a) providing a microemulsion comprising
(i) at least one alkyl (oligo)glycoside,
(ii) at least one ester of glycerol with at least one C₁₀-C₂₄ fatty acid,
(iii) at least one oil, which is different from (ii), and
(iv) water, and
b) mixing the microemulsion with a cosmetic carrier, which comprises at least one polyhydroxy compound of the following formula (I), 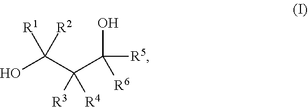
wherein
the residues R¹, R² independently of one another denote a hydrogen atom, a C₁-C₄ alkyl group or a phenyl group or in each case alkylene groups, which are constituents of a cyclopentane or cyclohexane ring,
the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring,
the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups,
with the proviso that the residues R³, R⁴ do not simultaneously denote a hydroxyl group.

2. The method according to claim 1, wherein in formula (I)
the residues R¹, R² independently of one another denote a hydrogen atom or a methyl group,
the residues R³, R⁴ independently of one another denote a hydrogen atom, a hydroxyl group or a methyl group, with the proviso that R³, R⁴ do not simultaneously denote a hydroxyl group,
the residues R⁵, R⁶ independently of one another denote a hydrogen atom or a methyl group, or one of the residues R⁵, R⁶ denotes a hydrogen atom or a methyl group and the other residue denotes one of the following groupings 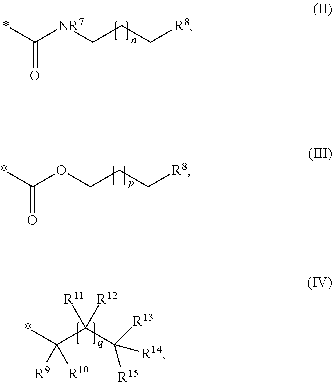
in which
R⁷ denotes a hydrogen atom or a C₁-₄ alkyl group,
R⁸ denotes a hydrogen atom, a methyl group or a hydroxyl group,
n, p, q independently of one another denote numbers from 0 to 10, and
the residues R⁹ to R¹⁵ independently of one another denote a hydrogen atom, a C₁-₄ alkyl group or a hydroxyl group, with the proviso that each C atom carries no more than one hydroxyl group.

3. The method according to claim 1, wherein the polyhydroxy compound is selected from 1,3-propylene glycol, glycerol, erythritol, pentaerythritol, sorbitol, mannitol, xylitol and/or panthenol.

4. The method according to claim 1, wherein the conditioning cleaning agent comprises—based on its total weight—0.01 to 10 wt. % of at least one polyhydroxy compound.

5. The method according to claim 1, wherein the microemulsion a) comprises
(i) 1 to 40 wt. % of at least one alkyl (oligo)glycoside of the general formula RO-[G]ₓ, in which R denotes an alkyl and/or alkenyl residue with 4 to 22 C atoms, G denotes a sugar residue with 5 or 6 C atoms and x denotes numbers from 1 to 10,
(ii) 1 to 15 wt. % of at least one saturated or unsaturated, branched or unbranched monoester and/or diester of glycerol with a C₁₀-C₂₄ fatty acid,
(iii) 5 to 45 wt. % of at least one oil, and
(iv) 40 to 80 wt. % water,
the quantitative data being based on the weight of the microemulsion a).

6. The method according to claim 1, wherein the conditioning cleaning agent comprises—based on its total weight—0.01 to 50 wt. % of the microemulsion a).

7. The method according to claim 1, wherein the cosmetic cleaning agent comprises—based on its total weight—0.1 to 40 wt. % of at least one anionic surfactant and 0 to 20 wt. % of at least one amphoteric/zwitterionic surfactant and/or 0 to 20 wt. % of at least one nonionic surfactant, the quantitative data not including the nonionic surfactants in the microemulsion a).

8. The method according to claim 1, wherein the conditioning cleaning agent comprises—based on its total weight—0.01 to 10 wt. % of a cationic polymer.

9. A conditioning cleaning agent, comprising
a) a microemulsion, which comprises
(i) at least one alkyl (oligo)glycoside,
(ii) at least one ester of glycerol with at least one C₁₀-C₂₄ fatty acid,
(iii) at least one oil, which is different from (ii), and
(iv) water, and
b) at least one polyhydroxy compound of the following formula (I), 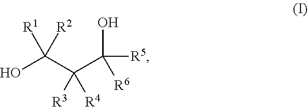
wherein
the residues R¹, R² independently of one another denote a hydrogen atom, a C₁-C₄ alkyl group or a phenyl group or in each case alkylene groups, which are constituents of a cyclopentane or cyclohexane ring,
the residues R³, R⁴ independently of one another denote a hydrogen atom, a hydroxyl group, a C₁-C₄ alkyl group, a hydroxyalkyl group, a phenyl group or in each case alkylene groups, which are constituents of a cyclopentane or cyclohexane ring, and
the residues R⁵, R⁶ independently of one another denote a hydrogen atom, a C₁-C₄ alkyl group or a hydroxyalkyl group, or one of the residues R⁵, R⁶ denotes a saturated or unsaturated, branched or unbranched C₁-C₂₀ alkyl chain, which can be interrupted by one or more ester and/or amide groupings and which can carry one or more hydroxyl groups,
with the proviso that the residues R³, R⁴ do not simultaneously denote a hydroxyl group.
